(19) United States
(12) Plant Patent Application Publication
(10) Pub. No.: US 2003/0005494 P1
(43) Pub. Date: Jan. 2, 2003

(54) Hybrid tea rose variety 'POULnew'

(76) Inventors: L. Pernille Olesen, Fredensborg (DK); Mogens N. Olesen, Fredensborg (DK)

Correspondence Address: Poulsen Roser Pacific, Inc., 620 S. Front Street, Central Point, OR, 97502, US

(21) Appl. No.: 10/155,852

(22) Filed: May 24, 2002

Related U.S. Application Data
(63) Continuation of application No. 09/277,241, filed on Mar. 26, 1999, now abandoned.

(51) Int. Cl. A01H 5/00

(52) U.S. Cl. PLT/133

(57) ABSTRACT

A new hybrid tea rose plant which has abundant, large, cream white, fragrant flowers and attractive, dark-green foliage. This new and distinct variety has shown to be uniform and stable in the resulting generations from asexual propagation.

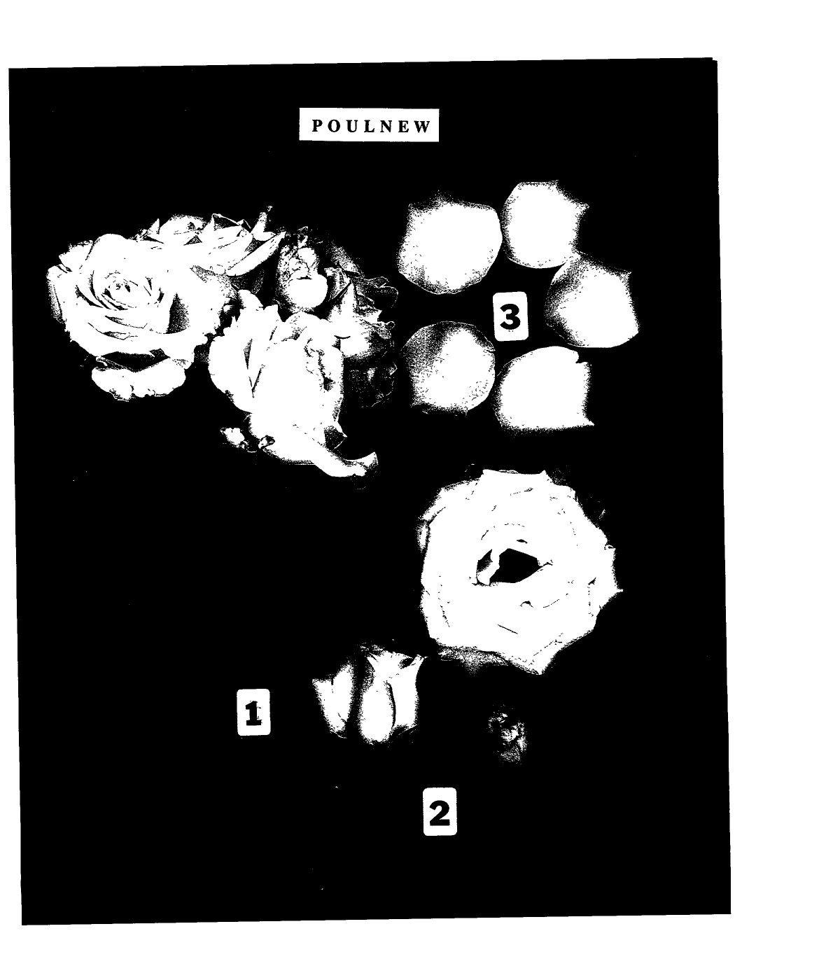

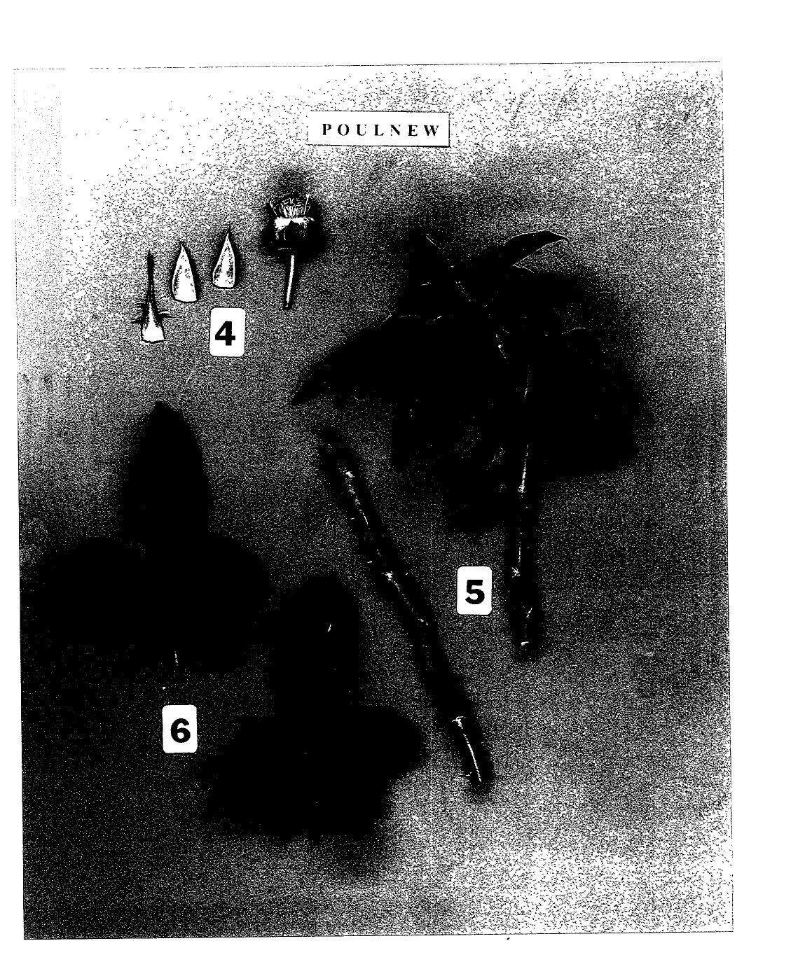

### SUMMARY OF THE INVENTION

The present invention constitutes a new and distinct variety of hybrid tea rose plant which originated from a controlled crossing between ‘Peauduce’ (non-patented) and an unnamed, non-commercialized seedling (non-patented). The two parents were crossed and the resulting seeds were planted in a controlled environment. The new variety is named ‘POULnew’.

The new rose may be distinguished from its seed parent, ‘Peauduce’, by the following combination of characteristics:

1. The seed parent has light yellow blooms; whereas, ‘POULnew’ has cream white blooms;

2. The growth habit of the seed parent is wider and more shrubby when compared to ‘POULnew’.

The new variety may be distinguished from its pollen parent, an unnamed seedling, created by the same inventors, by the following combination of characteristics:

1. The pollen parent is a climbing rose while ‘POULnew’ is shorter and shrubby;

2. The pollen parent has clear yellow blooms; whereas, ‘POULnew’ has cream white blooms;

3. The pollen parent has semi-double blooms compared to the double blooms of ‘POULnew’.

The objective of the hybridization of this rose variety for garden use was to create a new and distinct variety with unique qualities, such as:

1. Uniform and abundant flowers;

2. Vigorous and compact growth;

3. Fragrance;

4. Continuous blooming;

5. Disease resistance.

This combination of qualities is not present in previously available commercial cultivars of this type and distinguishes ‘POULtnew’ from all other varieties of which we are aware.

As part of their rose development program, L. Pernille Olesen and Mogens N. Olesen germinated the seeds from the aforementioned hybridization and conducted evaluations on the resulting seedlings in a controlled environment in Fredensborg, Denmark.

‘POULnew’ was selected in spring 1989 by the inventors as a single plant from the progeny of the aforementioned hybridization.

Asexual reproduction of ‘POULnew’ by traditional budding was first done by L. Pernille and Mogens N. Olesen in August 1989. This initial and other subsequent propagations conducted in controlled environments have demonstrated that the characteristics of ‘POULnew’ are true to type and are transmitted from one generation to the next.

### BRIEF DESCRIPTION OF THE DRAWING

The accompanying color illustration shows, as true as is reasonably possible to obtain in color photographs of this type, the typical characteristics of the buds, flowers, leaves, and stems of ‘POULnew’. Specifically illustrated in SHEET 1:

1. Stem showing branching and the attachment of leaves, buds, and peduncles;

2. Flower bud, partially opened bud, and open bloom;

3. Flower petals, detached;

Specifically illustrated in SHEET 2:

4. Sepals, receptacle, and pedicel;

5. Flowering stem as well as a bare stem exhibiting thorns;

6. Leaves.

### DETAILED DESCRIPTION OF THE VARIETY

The following is a description of ‘POULnew’, as observed in its growth in a field nursery in Jackson County, Oregon in September 1998, on plants eighteen months old. Color references are made using The Royal Horticultural Society (London, England) Colour Chart, 1995, except where common terms of color are used.

For a comparison, several physical characteristics of the rose variety ‘POULari’, a hybrid tea rose variety from the same inventors described and illustrated in U.S. Plant Pat. No. 9,274 and issued on September 5, 1995 are compared to ‘POULnew’ in Chart 1.

CHART 1

| | ‘POULnew’ | ‘POULari’ |
|---|---|---|
| Size of | 120 mm-130 mm. | 120-140 mm. |
| open bloom. | | |
| Petalage. | Double, 30-35 petals. | Very double, 55- |
| | | 75 petals. |
| Color of top | Yellow-White Group | White Group |
| of open bloom. | 158D to White Group 155D. | 155D. |

Parents:

Seed Parent.—‘Peauduce’.

Pollen Parent.—unnamed seedling.

Classification:

Botanical.—Rosa hybrida.

Commercial.—Hybrid Tea.

### FLOWER AND FLOWER BUD

Blooming habit: Nearly continuous.

Flower bud:

Size.—Upon opening, 35 mm−45 mm in length from base of receptacle to end of bud.

Bud form.—Pointed ovoid and high-centered.

Bud color.—As sepals unfold, Yellow-Orange Group 18B, graduating to Orange Group 27C. Blend of Orange Group 27C-D at ¼opening. Marginal zone of reverse side of petal with intonations of Red Group 39A.

Sepals.—Green Group 137C, graduating to Green Group 143B. Weak foliaceous appendages on three of the five sepals. Surfaces of sepals slightly pubescent.

Stipitate glands are present in limited numbers on margins of sepals. Sepals are 20-25 mm long and 10-12 mm wide. The apex shape is subulate.

Receptacle.—Surface: Smooth Shape: Funnel Size: Medium. 4-6 mm (h)×8-10 mm (w) Color: Green Group 138B.

Peduncle.—Surface: Smooth, glabrous Length: 50-60 mm average length Color: Green Group 143B-144B Strength: Strong.

Borne.—Generally with 3-4 buds per flowering stem.

Flower bloom:

Fragrance.—Strong, fruity, with smoky undertones.

Duration.—As a cut flower 5 to 6 days. The blooms have a duration on the plant of approximately 6 to 7 days. Petals fall cleanly away from plant.

Size.—Average flower diameter is 120-130 mm when open.

Form.—Open cup which reveals floral parts which are highly decorative.

Shape of flower when viewed from the side.—

Upon opening, upper part.—Convex.

Upon opening, lower part.—Convex.

Open flower, upper part.—Flattened Convex.

Open flower, lower part.—Flat.

Petalage.—Double, with 30-35 petals under normal conditions and 2-5 petaloids.

Color:

Upon opening, petals.—Upper Surface: Yellow-White Group 158D to White Group 155D becoming more yellow near basal zone. Reverse Side: Margin of reverse side on opening flower with intonations of Red Group 36A to Orange Group 27D. Middle zone of petal Yellow-White Group 158B-D. Color transitions in basal zone to Yellow Group 6D. Innermost petals: As above, but with greater intensity of yellow hue in middle and basal zone of petal.

Upon opening, basal petal spots.—Outermost petals: Outer Side: Yellow Group 5C. Inner Side: Yellow Group 5A. Innermost petals: Outer Side: Yellow Group 5B. Inner Side: Yellow Group 5A.

After opening, petals.—Petals: Upper Surface: Marginal zone, Yellow-White Group 158D. Middle zone and basal zone Yellow-White Group 158D to White Group 155D Reverse Side: Marginal zone Yellow-White Group 158D. Middle zone and basal zone Yellow-White Group 158D to White Group 155D.

After opening basal petal spots.—Outer Side: Yellow Group ID. Inner Side: Yellow Group 5A.

General Tonality: On open flower Yellow-White Group 158D to Orange-White Group 159D. No change in the general tonality at the end of the 3-4^{th} day. Afterwards, general tonality is Yellow-White Group 158D to White Group 155D.

Petals.—

Petal Reflex.—Petals reflex slightly

Petal Edge.—Entire; sometimes notched

Shape.—Round

Petaloids.—1-5 petaloids. Petaloids are 5 to 10 mm long and 4 to 6 mm wide. Petaloids are velvety in texture. Color is Orange-White Group 159D.

Thickness.—Thick.

Arrangement.—Imbricated.

Reproductive Organs:

Pollen.—Color: Yellow-Orange Group 14B. Quantity: Scant, sometimes medium quantities.

Pistils.—

Quantity.—25 to 30.

Length.—10 to 12 mm.

Anthers:

Size.—3 to 4 mm long.

Color.—

Immature.—Yellow Group 13C.

Mature.—Greyed-Orange Group 163A to Brown Group 200B.

Quantity.—20 to 25.

Filaments:

Color.—Yellow-Orange Group 17A with Yellow Group 1C.

Length.—8 to 10 mm.

Stigmas: At same position as anthers with a variable length.

Color.—Yellow-Green Group 145D.

Styles: Styles have fine, white hair covering entire length.

Color.—Green-White Group 157A. Other intonations: Below stigma, style is Greyed-Purple Group 185B.

Hips None observed.

### PLANT

Plant growth: Extremely vigorous. Upright. When grown as a budded field grown plant on multiflora understock, the average height of the plant itself is 150 cm and the average width is 75-80 cm.

Stems:

Color.—Young wood: Yellow-Green Group 144A Older wood: Green Group 138B

Thorns.—Incidence: 10 to 15 thorns per 10 cm of stem. Size: Average length: 4 mm. Color: Young: Red Group 36B. Mature: Greyed-Orange Group 174B. Shape: Concave.

Surface.—Young wood: Smooth. Older wood: Smooth.

Plant foliage: Quantity of leaflets on leaves in middle of the stem: 5 leaflets.

Leaf size.—Large. 140 mm (1) x 110 mm (w).

Quantity.—Average.

Color.—Upper Leaf Surface: Green Group 137A, graduating to Green Group 139A.

Lower Leaf Surface.—Green Group 138B.

Juvenile foliage.—Upper Surface, Green Group 137A. Lower Surface Greyed-Red Group 181B, with intonations of Green Group 137C.

Anthocyanin.—Location: Growth tip, juvenile leaves, prickles, inner sepals and tips, rachis. Color: Greyed-Red 181B.

Plant leaves and leaflets:

Stipules.—Size: 12 mm-16 mm. Color: Yellow-Green Group 144A. Stipitate glands: On margins of stipules.

Petiole.—Length: 25 mm - 35 mm. Color: Green Group 137B. Underneath: Small prickles on underside and a limited number of stipitate glands. Margins: Stipitate glands on upper surface.

Rachis.—Color: Green Group 137B. Underneath: Small prickles on underside and a limited number of stipitate glands. Margins: With stipitate glands on leaflet petioles.

Leaflet.—Edge: Serrated. Shape: Ovate. The leaflet's apex is acuminate. The leaflet's base is rounded. Arrangement: The leaflets are arranged in an odd- pinnate formation. Venation: The leaflets are veined in a reticulate pattern. Texture: Leaves are thick. Upper side of leaflet is moderately glossy. Lower side of leaflet is matte.

Disease resistance:

Average resistance to mildew, black spot, and Botrytis under normal growing conditions in Half Moon Bay, Califa., Jackson County, Oregon, Burlington, Canada, and Fredensborg, Denmark.

Winter hardiness:

‘POULnew’ has been found to be resistant to damage from cold in USDA Zone 8 and USDA Zone 7.

1. A new and distinct variety of rose plant of the hybrid tea class, substantially as herein illustrated and described as a distinct and novel rose variety due to its abundant, cream-white, fragrant flowers, vigorous and compact growth, and disease resistance:
